(19) United States
(12) Patent Application Publication
(10) Pub. No.: US 2021/0290824 A1
(43) Pub. Date: Sep. 23, 2021

(54) SURFACE FUNCTIONALIZED IMPLANT AND METHOD OF GENERATING THE SAME

(71) Applicant: New York Stem Cell Foundation, Inc., New York, NY (US)

(72) Inventors: Giuseppe Maria de Peppo, New York, NY (US); Hakan Engqvist, Uppsala (SE); Martina Sladkova, New York, NY (US)

(21) Appl. No.: 16/324,459

(22) PCT Filed: Aug. 10, 2017
(86) PCT No.: PCT/US2017/046336

§ 371 Date: Feb. 8, 2019

Related U.S. Application Data
(60) Provisional application No. 62/372,985, filed on Aug. 10, 2016.
(60) Provisional application No. 62/412,177, filed on Oct. 24, 2016.
(60) Provisional application No. 62/440,911, filed on Dec. 30, 2016.

(51) Int. Cl. A61L 27/38; A61L 27/36; A61L 27/58; A61L 27/22; A61L 27/24; A61L 27/52

(52) U.S. Cl. CPC A61L 27/3834; A61L 27/3633; A61L 27/365; A61L 27/3886; A61L 2430/02; A61L 27/222; A61L 27/24; A61L 27/52; A61L 2430/12; A61L 27/58

(57) ABSTRACT

The present invention provides a method for functionalizing a medical implant surface to promote osseointegration upon implantation in bone tissue.

### CROSS REFERENCE TO RELATED APPLICATIONS

This application claims the benefit of priority under 35 U.S.C. § 119(e) of U.S. Provisional Patent Application Ser. No. 62/372,985, filed Aug. 10, 2016, U.S. Provisional Patent Application Ser. No. 62/412,177, filed Oct. 24, 2016, and U.S. Provisional Patent Application Ser. No. 62/440,911, filed Dec. 30, 2016, the entire contents of which are incorporated herein by reference in their entireties.

### BACKGROUND INFORMATION

### Field of the Invention

The present invention relates generally to tissue engineering, and more particularly to a method of generating a surface functionalized medical implant.

### Background of the Invention

In dentistry and orthopedics, prosthetic implants are used to treat edentulous people and patients suffering from skeletal defects, with a global market worth billions of dollars each year. Titanium (Ti) is one of the most studied and used material in prosthetics due to its good biocompatibility, strong mechanical properties and ability, under favorable healing conditions, to form a structural and functional connection with bone. Osseointegration is dependent on the site of implantation as well as patient health, yet chemistry and topography of implant materials play a very critical role. Intense research is therefore ongoing to modify the surface of prosthetic implants and enhance their therapeutic potential. Traditional attempts to functionalize Ti implants include mechanical, physical, and chemical modifications of the implant surface. These modifications enhance the osteoconductivity of prosthetic implants but fail to actively regulate tissue response and healing. To modulate the surrounding tissue environment, facilitate osseointegration, and mitigate any potential adverse tissue response following implantation, researchers have recently explored the possibility to immobilize structural and biologically functional molecules on the surface of prosthetic devices, including Ti implants. While immobilization of biomolecules can lead to improved osseointegration, these modifications lack the degree of complexity typically found in human tissues at micro- and nano-scale, and may not be suited to treat at-risk patients with severely compromised healing ability. To further enhance the therapeutic potential of Ti implants, researchers have also combined these devices with stem cells with encouraging results. However, the use of live cells manipulated in the laboratory raises many safety concerns, with the cells potentially becoming ineffective or dangerous after implantation, and producing significant adverse effects such as tumors, aggressive immune reactions, or growth of undesired tissue.

Decellularization methods allow researchers to remove cells from tissue and organs while preserving the structural and functional mixture of proteins that constitute the extracellular matrix (ECM). Decellularized tissues have been successfully used for tissue engineering applications, because they display conductive and inductive properties that can modulate tissue response after implantation. In a similar fashion, decellularization methods open the possibility to functionalize the surface of prosthetic implants with biological cues of high molecular complexity at the micro- and nano-scale.

### SUMMARY OF THE INVENTION

Advances in stem cell biology, material science and engineering have facilitated the development of functional tissue substitutes using progenitor cells derived from induced pluripotent stem (iPS) cells. Building on these discoveries, the present invention is based in part on the finding that different decellularization treatments of implant surfaces seeded with mesenchymal progenitor (MP) cells derived from iPS cells (iPSC-MPs), result in diverse surface modifications, which affect proliferation and gene expression of the human iPSC-MPs. The decellularization protocols affect the expression of bone specific genes and opens unprecedented possibilities for development of personalized medical implants with enhanced osseointegration potential.

Accordingly, in one aspect, the invention provides a method for functionalizing a surface of an implant to promote osseointegration upon implantation into bone tissue. The method includes: a) seeding the surface of the implant with mesenchymal progenitor (MP) cells; b) culturing and expanding the cells to produce an extracellular matrix (ECM) on the surface; and c) decellularizing the surface, thereby functionalizing the surface of the implant. In embodiments, decellularizing removes cells from the implant surface while maintaining the ECM. For example, decellularization may be performed by contacting the surface with any agent, chemical reagent, or mechanical or physical process that is capable of lysing and removing cells from the implant surface. In some embodiments, decellularizing is accomplished by incubating the implant surface in a treatment solution including water, an alcohol, or a nonionic surfactant, or alternatively subjecting the implant surface to a freeze/thaw cycle in a physiological buffer such as phosphate-buffered saline (PBS). In some embodiments, decellularizing is accomplished by a physical or mechanical process which lyses and removes cells, such as high pressure and/or temperature sterilization, application of electromagnetic radiation, freezing, heating, high pressure fluid or gas, application of mechanical force such as scraping, and the like.

In various embodiments, the implant surface may be coated with one or more molecules that support adherence of a living cell before seeding, for example, coating with a polypeptide, gelatin, matrigel, entactin, glycoprotein, collagen, fibronectin, laminin, poly-D-lysine, poly-L-ornithine, proteoglycan, vitronectin, polysaccharide, hydrogel, or combinations thereof.

In various embodiments, seeded cells may be cultured for any amount of time. In some embodiments, the cells are cultured for at least about 2, 3, 4, 5, 6, 7, 8, 9, 10, 11, 12, 13, 14 days or longer, such as 21 or 28 days. In one embodiment, the cells are cultured for about 7 to 14 days.

In another aspect, the present invention provides a medical implant having a surface that is functionalized via the method of the disclosure.

In yet another aspect, the invention provides a hybrid bone implant which includes a surface functionalized implant of the disclosure and a bone tissue graft having a three dimensional scaffold. The scaffold may be engineered and be generated from iPS cells derived from a subject whom will be the recipient of the hybrid implant.

In still another aspect, the invention provides a method for performing a medical procedure. The method includes: a) obtaining a cell from a subject; b) reprogramming the cell of (a) to generate an inducted pluripotent stem (iPS) cell; c) differentiating and expanding the iPS cell to generate mesenchymal progenitor (MP) cells; d) seeding the MP cells onto the surface of an implant; e) culturing and expanding the MP cells to produce an extracellular matrix (ECM) on the surface; f) decellularizing the surface, thereby functionalizing the surface of the implant to promote osseointegration upon implantation into bone tissue; and g) implanting the implant into bone tissue of the subject.

### BRIEF DESCRIPTION OF THE FIGURES

FIG. 1 is a schematic diagram illustrating the methodology for generating an implant in one embodiment of the invention.

FIGS. 2A-2D are a series of graphic and pictorial representations showing titanium (Ti) disk implant characterization in one embodiment of the invention. FIG. 2A is a photograph of a Ti disk implant. FIG. 2B is series of SEM images of a Ti disk implant taken at 1000× (scale bar=50 μm), 5000× (scale bar=10 μm), and 15,000× (scale bar=1 μm) magnification. FIG. 2C depicts 3D morphological analysis of a Ti disk with values of surface roughness (Ra, Rq and Rt). FIG. 2D depicts EDS analysis of a Ti disk showing the surface elemental composition (scale bar=25 μm).

FIG. 3 is a series of images showing cell seeding (day 2) and expansion (day 14) of an implant in one embodiment of the invention.

FIG. 4A is a series of SEM images of the surface of implants treated with various decellularization protocols.

FIG. 4B is a series of SEM images of the surface of implants treated with various decellularization protocols.

FIG. 4C is a series of graphs depicting EDS analysis of implants treated with various decellularization protocols.

FIG. 5 is a graph depicting cell growth data on an implant in embodiments of the invention.

FIG. 6A is a series of graphs depicting nanostring analysis of reseeded implants in embodiments of the invention.

FIG. 6B is a clustering graph depicting nanostring analysis of reseeded implants in embodiments of the invention.

FIG. 7A is a series of graphs depicting gene expression of cultured cells on implants in embodiments of the invention.

FIG. 7B is a series of graph depicting alkaline phosphatase levels of control and reseeded implant samples.

FIG. 8 is a diagram setting forth gene expression analysis in embodiments of the invention.

FIG. 9 is a graph depicting XPS analysis.

FIG. 10 is a graph depicting alkaline phosphatase levels of control and reseeded implant samples.

FIG. 11 is a schematic depicting decellularization protocols in embodiments of the invention.

FIG. 12 is a schematic depicting a reseeding protocol in one embodiment of the invention.

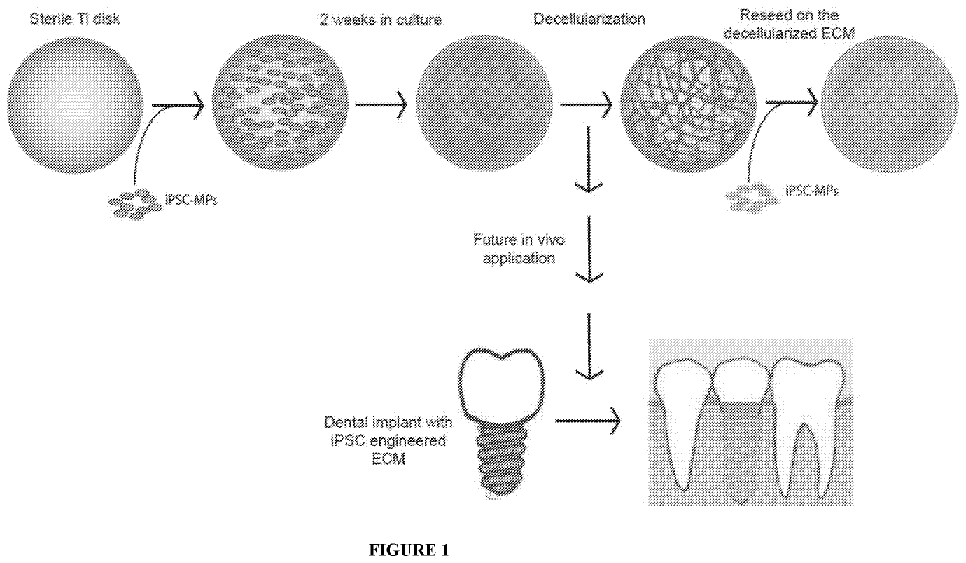

### DETAILED DESCRIPTION OF THE INVENTION

The present invention provides a method for functionalizing a medical implant surface to promote osseointegration upon implantation in bone tissue.

Titanium implants are widely used in dentistry and orthopedics because they can form a stable bond with surrounding bone following implantation, a process known as osseointegration. Yet, full integration of prosthetic implants takes time, and often fails in clinical situations characterized by poor bone quality, compromised regenerative capacity, and other factors that are still unclear (i.e. diabetic patients). Intense research efforts are thus made to develop new implants that are cost-effective, safe, and optimal for each patient in each clinical situation.

As disclosed in the Example set forth herein, the inventors tested the possibility of functionalizing the surface of Ti implants using stem cells. Human induced pluripotent stem cell-derived mesenchymal progenitor (iPSC-MP) cells were cultured on Ti model disks for 2 weeks in osteogenic conditions. The samples were then decellularized using four different decellularization methods, including treatment with deionized water, ethanol, freeze-thaw cycles, and triton to wash off the cells and expose the matrix. Following treatment, the samples were characterized using scanning electron microscopy (SEM), energy-dispersive X-ray spectroscopy (EDS) and X-ray photoelectron spectroscopy (XPS) to examine and compare the decellularization potential of each method. Finally, the functionalized samples were sterilized and seeded with fresh human iPSC-MP cells to investigate the effect of stem cell-mediated functionalization of Ti implants on cell proliferation, gene expression, and differentiation.

The results show that different decellularization treatments result in diverse surface modifications, which affect proliferation and gene expression of human iPSC-MPs. Interestingly, the decellularization protocols affected the expression of bone specific genes, suggesting that ECM affects stem cell differentiation. Cell-mediated functionalization represents an interesting route to modify the surface of Ti implants with cues of biological relevance, and opens unprecedented possibilities for development of personalized implants with enhanced osseointegration potential.

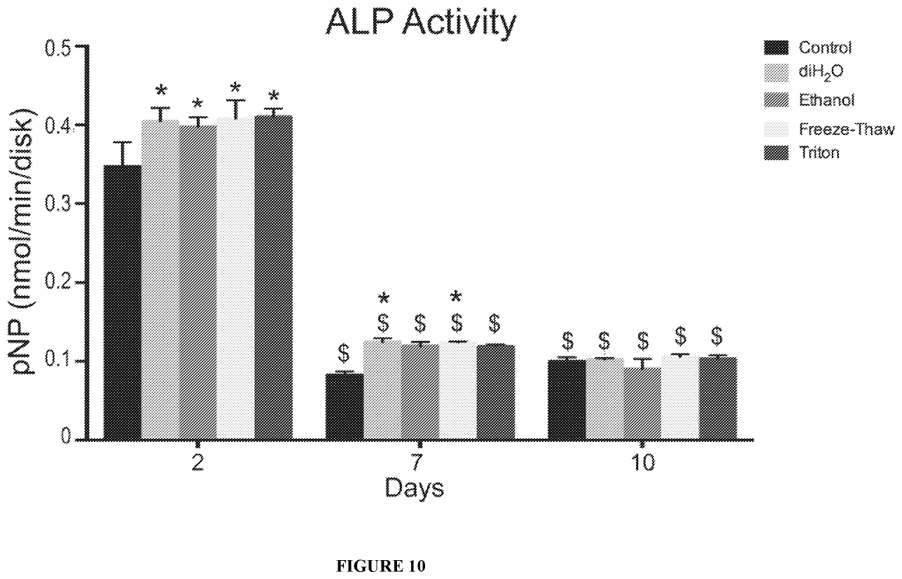

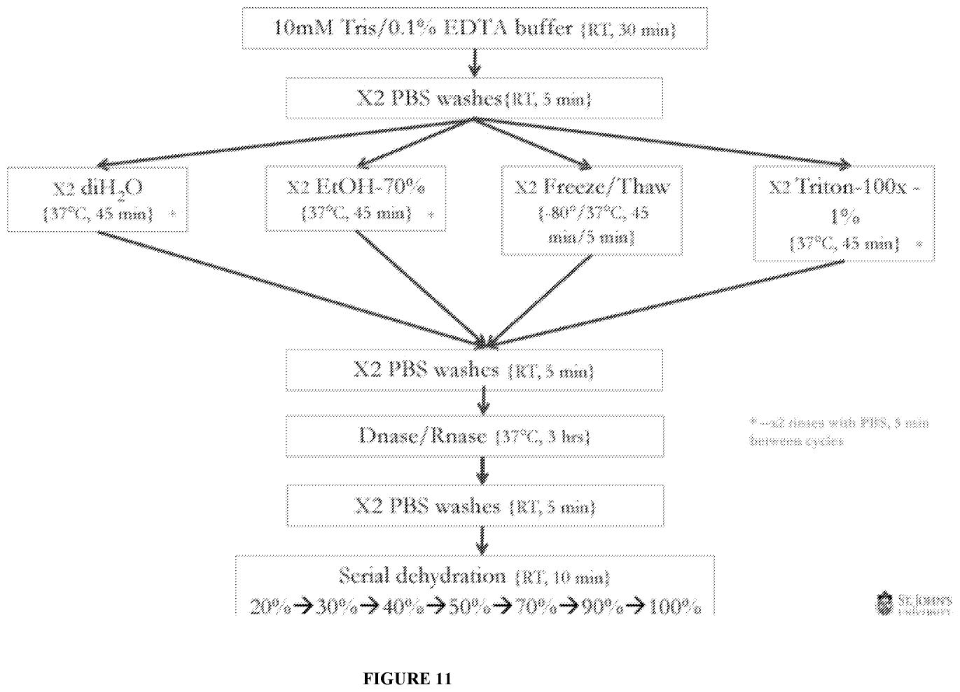

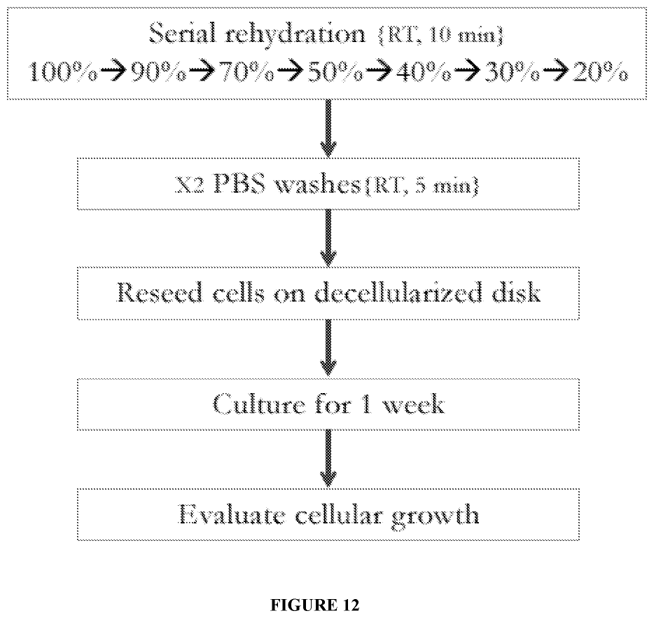

Accordingly, in one aspect, the invention provides a method for functionalizing a surface of an implant to promote osseointegration upon implantation into bone tissue. The method includes: a) seeding the surface of the implant with mesenchymal progenitor (MP) cells; b) culturing and expanding the cells to produce an extracellular matrix (ECM) on the surface; and c) decellularizing the surface, thereby functionalizing the surface of the implant. In embodiments, decellularizing removes cells from the implant surface while maintaining the ECM. Decellularizing is accomplished by incubating the implant surface in a treatment solution including water, an alcohol, or a nonionic surfactant, or alternatively subjecting the implant surface to a freeze/thaw cycle in a physiological buffer such as phosphate-buffered saline (PBS). In various embodiments, decellularization includes a protocol as set forth in FIG. 10. For example, decellularization may include incubation of the surface in a treatment solution, wherein the solution includes deionized water, ethanol or a nonionic surfactant, such as Triton™ X-100. Typically, the incubation is performed at about 37° C. for up to or greater than 10, 20, 30, 40, 50, 60, 70, 80 or 90 minutes. After incubation, the surface may be washed with a physiological buffer, such as PBS.

In embodiments, the implant surface may be treated with a nuclease after incubation with the treatment solution. In embodiments, the surface is treated with a DNAse, RNAse, or combination thereof

As will be appreciated, the implant may be composed of a variety of materials with are suitable for implantion into a patient. As number of a biocompatible materials are well known in the art and suitable for use with the present invention. In one embodiment, the implant is composed of a biocompatible metal or alloy, such as titanium, aluminum alloy, nickel alloy, titanium alloy, cobalt-chrome alloy or medical grade steel.

Non-limiting examples of various implant materials include de-cellularized tissue (such as de-cellularized bone) and natural or synthetic polymers or composites (such as ceramic/polymer composite materials). In some embodiments the implant material may be capable of being absorbed by cells (e.g., resorbable materials), while in other embodiments non-resorbable implant materials may be used. In some embodiments, the implant may comprise, consist of, or consist essentially of, any of the above-listed materials, or any combination thereof.

In one embodiment, the invention provides a hybrid bone implant which includes a surface functionalized implant of the disclosure and a bone tissue graft having a three dimensional scaffold. In one embodiment, the functionalized implant material is a biocompatible metal, such as titanium or medical grade steel, and the tissue graft comprises a scaffold material including natural or synthetic bone.

In various embodiments, scaffolds can be made of any suitable material having appropriate pore sizes, porosity and/or mechanical properties for the intended use. Such suitable materials will typically be non-toxic, biocompatible and/or biodegradable, and capable of infiltration by cells of the desired tissue graft type, for example bone-forming cells in the case of bone tissue grafts. Non-limiting examples of such materials include de-cellularized tissue (such as de-cellularized bone), materials that comprise or one or more extracellular matrix (“ECM”) components such as collagen, laminin, and/or fibrin, and natural or synthetic polymers or composites (such as ceramic/polymer composite materials). In some embodiments the scaffold material may be capable of being absorbed by cells (e.g., resorbable materials), while in other embodiments non-resorbable scaffold materials may be used. In some embodiments, the scaffold may comprise, consist of, or consist essentially of, any of the above-listed materials, or any combination thereof.

In some embodiments, the dimensions and geometry of a scaffold correspond to that of a three-dimensional model, such as a digital model, of a tissue portion. In some embodiments the dimensions and geometry of a scaffold can be designed or selected based on such a model in order to facilitate culturing of cells, e.g., tissue-forming cells or other cells as described herein, on the scaffold within a bioreactor, as further described in International Application Nos. PCT/US2016/25601 and PCT/US2015/064076, incorporated herein by reference in their entireties. This may be done, for example, to produce a tissue graft or tissue graft segment having a size and shape corresponding to that of a three-dimensional model.

In some embodiments, the scaffold is generated as described in International Application Nos. PCT/US2016/25601 and PCT/US2015/064076. For example, the scaffold may be generated or customized using computer-assisted manufacturing. For example, a tissue model segment file can be used with, CAM software to drive the fabrication of geometrically defined scaffolds using any suitable method known in the art, or a combination thereof, for example, computer-controlled milling methods, rapid prototyping methods, laser cutting methods, three-dimensional printing, and/or casting technologies. In some embodiments, manufacturing of the scaffold comprises using rapid prototyping, a milling machine, casting technologies, laser cutting, and/or three-dimensional printing, or any combination thereof. In some embodiments, manufacturing of the scaffold comprises using computer-numerical-control, such as when the manufacturing comprises laser cutting or using a milling machine. For example, digital models, such as those generated using CAD software as described above, can be processed to generate the appropriate codes (such as “G-Codes”) to drive a computer-numerical-control (CNC) milling machine (for example, Tormach™, Bridgeport™) and to select appropriate machining tool bits and program machining paths to cut the scaffold material into the desired shapes and sizes (e.g., corresponding to that of a digital models of a tissue segment).

While scaffolds provided by the invention can be designed and manufactured as described herein, a person having ordinary skill in the art will appreciate that a variety of other methods of designing and manufacturing may be used to generate scaffolds according to the present invention.

In some embodiments scaffolds are engineered from induced pluripotent stem cells using a biomimetic approach of bone development in vitro (de Peppo et al., PNAS 110(21):8680-5 (2013); and International Application Nos. PCT/US2016/25601 and PCT/US2015/064076).

Any suitable or desired type of cell or cells may be used in the present invention. Typically, to functionalize an implant surface, mesenchymal progenitor (MP) cells are used. In embodiments, the MP cells are generated from iPS cells produced from cells isolated from a patient. Further, cell types may be combined to produce tissue grafts, such as the hybrid bone implant of the invention. Typically the selected cell(s) will be capable of forming the desired tissue graft (for example, for a vascularized bone graft, mesenchymal progenitor cells and endothelial progenitor cells or any other cell types suitable for or capable of forming bone and blood vessels, as further described herein), or any cell(s) capable of differentiating into the desired tissue-forming cell(s) (for example, a pluripotent cell). Non-limiting examples of cells that may be used include pluripotent cells, stem cells, embryonic stem cells, induced pluripotent stem cells, progenitor cells, tissue-forming cells, or differentiated cells.

The cells used may be obtained from any suitable source. In some embodiments, the cells may be human cells. In some embodiments, the cells may be mammalian cells, including, but not limited to, cells from a non-human primate, sheep, or rodent (such as a rat or mouse). For example, cells may be obtained from tissue banks, cell banks or human subjects. In some embodiments, the cells are autologous cells, for example, cells obtained from the subject into which the prepared tissue graft will be subsequently transplanted, or the cells may be derived from such autologous cells. In some embodiments, the cells may be obtained from a “matched” donor, or the cells may be derived from cells obtained from a “matched” donor. For cell and tissue transplants, donor and recipient cells can be matched by methods well known in the art. For example, human leukocyte antigen (HLA) typing is widely used to match a tissue or cell donor with a recipient to reduce the risk of transplant rejection. HLA is a protein marker found on most cells in the body, and is used by the immune system to detect cells that belong in the body and cells that do not. HLA matching increases the likelihood of a successful transplant because the recipient is less likely to identify the transplant as foreign. Thus, in some embodiments of the present invention, the cells used are HLA-matched cells or cells derived from HLA-matched cells, for example, cells obtained from a donor subject that has been HLA-matched to the recipient subject who will receive the tissue graft. In some embodiments the cells used may be cells that have been modified to avoid recognition by the recipient's immune system (e.g. universal cells). In some such embodiments the cells are genetically-modified universal cells. For example, in some embodiments the universal cells may be MHC universal cells, such as major histocompatibility complex (MHC) class I-silenced cells (see, i.e., Figueiredo et al., Biomed Res Int (2013)). Human MHC proteins are referred to as HLA because they were first discovered in leukocytes. Universal cells have the potential to be used in any recipient, thus circumventing the need for matched cells.

In some embodiments, the cells used in practicing the invention are, or include, pluripotent stem cells, such as induced pluripotent stem cells (iPSCs). In some such embodiments, the pluripotent stem cells may be generated from cells obtained from the subject (i.e. autologous cells) that will receive the implant. In other such embodiments, the pluripotent stem cells may be generated from cells obtained from a different individual—i.e. not the subject that will receive the implant (i.e. allogeneic cells). In some such embodiments, the pluripotent stem cells may be generated from cells obtained from a different individual—i.e. not the subject that will receive the tissue graft—but where that different individual is a “matched” donor—for example as described above. In some embodiments, the cells used are differentiated cells, such as bone cells. In some embodiments, the differentiated cells are derived from pluripotent stem cells, such as induced pluripotent stem cells. In some embodiments, the differentiated cells are derived by trans-differentiation of differentiated somatic cells, or by trans-differentiation of pluripotent cells (such as pluripotent stem cells or induced pluripotent stem cells), for example induced pluripotent stem cells generated from somatic cells.

A pluripotent stem cell is a cell that can (a) self-renew and (b) differentiate to produce cells of all three germ layers (i.e. ectoderm, mesoderm, and endoderm). The term “induced pluripotent stem cell” encompasses pluripotent stem cells, that, like embryonic stem cells (ESC), can be cultured over a long period of time while maintaining the ability to differentiate into cells of all three germ layers, but that, unlike ES cells (which are derived from the inner cell mass of blastocysts), are derived from somatic cells, that is, cells that had a narrower, more defined potential and that in the absence of experimental manipulation could not give rise to cells of all three germ layers. iPSCs generally have an hESC-like morphology, growing as flat colonies with large nucleo-cytoplasmic ratios, defined borders and prominent nuclei. In addition, iPSCs generally express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, iPSCs, like other pluripotent stem cells, are generally capable of forming teratomas. In addition, they are generally capable of forming or contributing to ectoderm, mesoderm, or endoderm tissues in a living organism.

Illustrative iPSCs include cells into which the genes Oct-4, Sox-2, c-Myc, and Klf have been transduced, as described by Takahashi and Yamanaka (Cell 126(4):663-76 (2006), the contents of which is hereby incorporated by reference in its entirety). Other exemplary iPSCs are cells into which OCT4, SOX2, NANOG, and LIN28 have been transduced (Yu et al., Science 318:1917-1920 (2007), the contents of which is hereby incorporated by reference in its entirety). One of skill in the art would know that various different cocktails of reprogramming factors can be used to produce iPSCs, such as factors selected from the group consisting of OCT4, SOX2, KLF4, MYC, Nanog, and Lin28. The methods described herein for producing iPSCs are illustrative only and are not intended to be limiting. Rather any suitable methods or cocktails of reprogramming factors known in the art can be used. In embodiments where reprogramming factors are used, such factors can be delivered using any suitable means known in the art. For example, in some embodiments any suitable vector, such as a Sendai virus vector, may be used. In some embodiments reprogramming factors may be delivered using modified RNA methods and systems. A variety of different methods and systems are known in the art for delivery of reprogramming factors and any such method or system can be used.

Any culture medium suitable for culture of cells, such as pluripotent stem cells, may be used in accordance with the present invention, and several such media are known in the art. For example, a culture medium for culture of pluripotent stem cells may comprise Knockout™ DMEM, 20% Knockout™ Serum Replacement, nonessential amino acids, 2.5% FBS, Glutamax, beta-mercaptoethanol, 10 ng/microliter bFGF, and antibiotic. The employed medium may also be a variation of this medium, for example without the 2.5% FBS, or with a higher or lower % of knockout serum replacement, or without antibiotic. The employed medium may also be any other suitable medium that supports the growth of human pluripotent stem cells in undifferentiated conditions, such as mTeSR™ (available from STEMCELL Technologies), or Nutristem™ (available from Stemgent™), or ES medium, or any other suitable medium known in the art. Other exemplary methods for generating/obtaining pluripotent stem cells from a population of cells obtained from a subject are provided in the Examples of the present application.

In some embodiments, pluripotent stem cells are differentiated into a desired cell type, for example, a bone-forming cell, or any other desired cell type. Differentiated cells provided by the invention can be derived by various methods known in the art using, for example, adult stem cells, embryonic stem cells (ESCs), epiblast stem cells (EpiSCs), and/or induced pluripotent stem cells (iPSCs; somatic cells that have been reprogrammed to a pluripotent state). Methods are known in the art for directed differentiation or spontaneous differentiation of pluripotent stem cells, for example by use of various differentiation factors. Differentiation of pluripotent stem cells may be monitored by a variety of methods known in the art. Changes in a parameter between a stem cell and a differentiation factor-treated cell may indicate that the treated cell has differentiated. Microscopy may be used to directly monitor morphology of the cells during differentiation.

In each of the embodiments of the invention, any suitable or desired types of cells can be used to produce the implant and/or scaffolds described herein, including, but not limited to, pluripotent stem cells or progenitor cells or differentiated cells. In some embodiments, the pluripotent stem cells may be induced pluripotent stem cells. In embodiments where induced pluripotent stem cells are used, such cells may be derived from differentiated somatic cells obtained from a subject, for example by contacting such differentiated somatic cells with one or more reprogramming factors. In some embodiments, pluripotent cells may have been induced toward a desired lineage, for example, mesenchymal lineage or endothelial lineage. In some embodiments, the differentiated cells can be any suitable type of differentiated cells. In some embodiments, the differentiated cells may be derived from pluripotent stem cells (such as induced pluripotent stem cells), for example by contacting such pluripotent cells with one or more differentiation factors. In some embodiments, the differentiated cells may be derived by trans-differentiation of another differentiated cell type, for example by contacting the cells with one or more reprogramming factors. In the various embodiments of the present invention involving differentiated cells, such differentiated cells may be any desired differentiated cell type, including, but not limited to, bone cells and blood vessel cells.

Any suitable or desired type of cell, such as the cell types described herein, can be applied to or seeded onto an implant surface or scaffold to prepare an implant or hybrid implant according to the present invention.

In some embodiments, cells that produce and deposit ECM, such as iPSC-MPs are used to seed the implant or scaffold. To promote cell adhesion, the surface of the implant or scaffold may be coated with at least one molecule that modifies the surface, for example to support adherence or growth of a living cell before seeding the surface with cells. Such molecules include by way of illustration, hyaluronic acid (HA), calcium phosphate, polypeptides, gelatin, matrigel, entactin, glycoprotein, collagen, fibronectin, laminin, poly-D-lysine, poly-L-ornithine, proteoglycan, vitronectin, polysaccharide, hydrogel, and combinations thereof.

In various embodiments, the surface of the implant is treated prior to seeding with cells. In one embodiment, the surface is treated to modify the surface. This can be accomplished by applying a surface modification agent or technique. In this manner one or more of the following properties may be modified: roughness, hydrophilicity, surface charge (impart positive or negative charge), surface energy, biocompatibility and reactivity.

In some embodiments, cells may be in a differentiated state prior to being applied to a scaffold. For example, in some embodiments differentiated cells may be obtained and used directly. Similarly, in some embodiments non-differentiated cells may be cultured according to any suitable method known in the art, such as in a culture dish or multi-well plate or in suspension, for a suitable period or length of time, for example, until desired levels of cell growth or differentiation or other parameters are achieved, then the differentiated cells may be transferred to the scaffold and subsequently the cell/scaffold construct is inserted into a bioreactor to facilitate development of a tissue graft. In some embodiments, non-differentiated cells (for example, stem cells (such as iPSCs) or progenitor cells) may be applied to the scaffold. In such embodiments, the non-differentiated cells may undergo differentiation while being cultured on the scaffold.

In some embodiments, two or more different cell populations may be seeded onto a scaffold to prepare a cell/scaffold construct. In some embodiments, the two or more populations of cells are co-cultured on the scaffold for a suitable period of time, for example, until desired levels of growth or differentiation or other parameters are achieved, before the cell/scaffold construct is inserted into the bioreactor. Populations of cells may comprise, consist essentially of, or consist of, any desired type of cell in any stage of growth or differentiation, and any combinations thereof. For example, in some embodiments, each cell population may comprise cells capable of forming a different tissue, for example for the preparation of a vascularized bone graft, a first population containing cells capable of forming bone, such as mesenchymal progenitor cells, and a second population containing cells capable of forming blood vessels, such as endothelial progenitor cells. In some embodiments, each population of cells may comprise cells capable of forming the same tissue (e.g., bone) but each population of cells may be at different stages of differentiation (e.g., mesenchymal stem cells and bone marrow stromal cells). Populations of cells to be co-cultured may be applied to a scaffold at the same time or at different times, as desired. Where two or more populations of cells are applied at different times, the sequence or order of co-culture (e.g., which population is applied to the scaffold first, which population is applied to the scaffold second, etc.) may be selected as desired, for example depending on the cell types being used, the state or growth or differentiation of the populations of cells, or any other parameters, as desired. Where two or more populations of cells are to be applied to the scaffold, they can be applied at any suitable cell ratio, as desired. For example, in some embodiments two different populations of cells may be seeded at a ratio of about 1:1, or any ratio from about 2:8 to about 8:2. In some embodiments, the cell populations may be seeded at a ratio of about 2:8, about 3:7, about 4:6, about 5:5, about 6:4, about 7:3, or about 8:2.

A person having ordinary skill in the art will recognize that countless variations and combinations of cells and culture methods will fall within the scope of the present invention. For example, cell culture methods, including cell seeding ratios, concentration of differentiation factors and sequence of co-culture, will typically be determined according to the desired cell type being used or the tissue graft being prepared.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE 1

### Generation of Medical Implant

Prosthetic implants are used daily to treat edentulous people and to restore mobility in patients affected by skeletal defects. Titanium (Ti) is the material of choice in prosthetics, because it can form a stable bond with the surrounding bone following implantation—a process known as osseointegration. Yet, full integration of prosthetic implants takes time, and fails in clinical situations characterized by limited bone quantity and/or compromised regenerative capacity, and in at-risk patients. Intense research efforts are thus made to develop new implants that are cost-effective, safe, and suited to every patient in each clinical situation. In this study, the possibility to functionalize Ti implants using stem cells was investigated. Human induced pluripotent stem cell-derived mesenchymal progenitor (iPSC-MP) cells were cultured on Ti model disks for 2 weeks in osteogenic conditions. Samples were then treated using four different decellularization methods to wash off the cells and expose the matrix. The functionalized disks were finally sterilized and seeded with fresh human iPSC-MP cells to study the effect of stem cell-mediated surface functionalization on cell behavior. The results show that different decellularization methods produce diverse surface modifications, and that these modifications promote proliferation of human iPSC-MP cells, affect the expression of genes involved in development and differentiation, and stimulate the release of alkaline phosphatase. Cell-mediated functionalization represents an attractive strategy to modify the surface of prosthetic implants with cues of biological relevance, and opens unprecedented possibilities for development of new devices with enhanced therapeutic potential.

Materials and Methods

Manufacturing and Characterization of Titanium Disks.

Titanium disks (area: 0.694 cm², thickness 0.5 mm) were punched out of flat sheets (Grade 2 titanium, Edstraco, Sweden) using an Amada CNC punch with 9 mm punching tools with associated cushion. After rinsing in 70% ethanol (v/v), the samples were sonicated in acetone for 30 min, and characterized to study the surface properties via SEM, profilometry, EDS, and XPS. For SEM analysis, samples were imaged using the FEI Helios NanoLab™ 660 (FEI, Hillsboro, Oreg.) with the following settings: 10 kV, 0.8 nA, and a working distance of 5.4 mm. Surface profile and roughness were measured using a Wyko T1100™ profilometer VSI mode (Bruker, Billerica, Mass.) with Vision software at 0.5× magnification. Surface chemistry was studied using the FEI Helios NanoLab™ 660 (FEI) with supplemental AZtec™ software at 10 keV with a dead time ranging from 40-60%. The XPS data were acquired at take-off angles of 45°. Multipak (Physical Electronics, Inc. (PHI)) was used for further quantitative chemical analysis. The C 1 s, N1s, O1 s, P2p and Ca2p spectra were acquired from an area of size 200×200 μm.

Preparation of Titanium Disks.

Prior to seeding, the disks were placed in sterilization pouches (Fisher Scientific, Pittsburgh, Pa.) and autoclaved. Using sterile forceps, the disks were then placed in 24-well plates (Thermoscientific, Roskilde, Denmark), and conditioned overnight at 37° C. with 1 ml of expansion medium consisting of high-glucose KnockOut™ Dulbecco's Modified Eagle's Medium (KO-DMEM; Gibco, Grand Island, N.Y.), 10% (v/v) HyClone fetal bovine serum (GE Life Sciences, Pittsburgh, Pa.), beta-fibroblast growth factor (1 ng/ml; R&D systems, Minneapolis, Minn.), GlutaMax™ (1×; Gibco), non-essential amino acids (1×; Gibco), 0.1 mM β-mercaptoethanol (Gibco), and antibiotic-antimycotic (1×; Gibco). Following this treatment, the disks were blotted on sterile Kimwipes (Roswell, Ga.) and air-dried for approximately 10 min. To enhance cell attachment, the disks were treated for 90 min with gelatin (0.1%; EmbryoMax®, Millipore, Billerica, Mass.) at 37° C. Before cell seeding, the disks were blotted to remove any excess gelatin and transferred to new 24-well plates.

Cell Seeding on Ti Disks.

Human iPSC-derived mesenchymal progenitor cells (line 1013A) were derived as previously described [27]. Before seeding, cells were expanded on gelatin (0.1%; EmbryoMax®, Millipore)-coated plasticware in expansion medium, then detached with trypsin/EDTA (0.25%; Thermoscientific), centrifuged and resuspended at a density of 2 × 10⁴ cells/ml. One ml of cell suspension was then added to each disk. After 2 days in expansion medium, the samples were cultured in osteogenic medium consisting of high-glucose DMEM (Gibco) supplemented with 10% (v/v) HyClone™ fetal bovine serum (GE Life Sciences), L-ascorbic acid (50 μM; Sigma-Aldrich, St Louis, Mo.), dexamethasone (1 μM; Sigma-Aldrich), and β-glycerophosphate disodium salt (10 mM; Sigma-Aldrich) for additional 12 days.

Cell Growth on Ti Disks.

Cell distribution and growth on Ti disks was monitored using the Live/Dead assay (Life Technologies, Frederick, Md.). Two days and two weeks after seeding, samples were stained with calcein (5 μl calcein in 10 ml of PBS) at 37° C. for 35 min. Following incubation, ethidium bromide was added to each well (2 μl per 1 ml of PBS) and sample incubated for additional 10 min. After incubation, samples were transferred to no phenol red Roswell Park Memorial Institute medium 1640 (RPMI; Gibco), and imaged via epifluorescence and confocal microscopy. To create mosaic images, sequential fluorescence images were taken at 4× magnification with an Olympus IX71 microscope (Olympus, Tokyo, Japan) equipped with the imaging program Olympus DP2-BSW. In order to obtain images of the whole samples, mosaic pictures were assembled from sequential fluorescent images using ImageJ™ (National Institute of Health) equipped with the Mosaic^{T}J™ and TurboReg™ plugins. Confocal images were taken with a Zeiss™ microscope (Zeiss, Oberkochen, Germany) using the Zen 9000™ computer platform.

Sample Decellularization.

After 14 days in culture, samples were harvested and treated to lyse and remove the cells using 4 different decellularization protocols. Before treatment, all samples were treated in a 24-well plate with a 10 mM Tris buffer solution containing 0.1% EDTA (w/v) for 30 min at room temperature (RT). Samples were then washed with PBS twice for 5 min at RT, and decellularized using the protocols as described below.

Distilled water: samples were immersed in 1 ml of sterile distilled H2O, and incubated at 37° C. for 45 min on a Benchrocker™ 2D tilter (Benchmark Scientific, Sayreville, N.J.). After the first cycle, samples were washed with PBS for 5 min at RT, and then immersed again in 1 ml of sterile distilled H₂O for a second decellularization cycle;

Ethanol: samples were immersed in 1 ml of 70% ethanol (v/v) and incubated at 37° C. for 45 min on a Benchrocker™ 2D tilter as described above. After the first cycle, the samples were washed with PBS for 5 min at RT, and then immersed again in 1 ml of 70% ethanol (v/v) for a second decellularization cycle.

Freeze/thaw: samples were immersed in 1 ml of PBS, and placed at −80° C. for 45 min. Afterward, the samples were allowed to thaw at 37° C. for 20 min. The freeze/thaw cycle was repeated twice with samples immersed in 1 ml of PBS.

Triton: samples were immersed in 1 ml of 0.01% (v/v) triton (100×; Sigma), and incubated at 37° C. for 45 min on a Benchrocker™ 2D tilter as described above. After the first cycle, the samples were washed with PBS for 5 min at RT, and then immersed again in 1 ml of 0.01% (v/v) triton for a second decellularization cycle.

Following treatment, all samples were washed with PBS twice for 5 min and dehydrated for characterization.

Characterization of Functionalized Titanium Disks.

Before characterization, samples were dehydrated using ethanol solutions with increasing concentration, i.e. 20, 40, 50, 70, 90 and 100% (v/v) for 10 min each. Samples were then air-dried and characterized via SEM, EDS, and XPS to examine the decellularization potential of each method. Non-decellularized samples were fixed overnight in paraformaldehyde 4% (v/v) at 4° C. and used as controls. For SEM analysis, samples were sputtered with a 10-50 nm gold layer to increase sample conductivity, then imaged using the FEI Helios NanoLab™ 660 (FEI) with 5-10 kV voltages, a 0.8 nA current, and a working distance between 4.9-5.4 mm. For lateral imaging of the samples, dehydrated samples were incubated in 100% isopropanol twice for 15 min, cleared in xylene twice for 15 min, and embedded in polymethyl methacrylate resin using the EMBed-812 kit (Electron Microscopy Sciences, Hatfield, Pa.) according to the manufacturer's instructions, and sectioned vertically using a slow-speed saw (Isomet, Buehler, Lake Bluff, Ill., USA). Sections of approximately 300 pm thickness were sputter-coated with 10 nm gold layer and imaged using SEM at following settings: 3kV, 80 pA, and working distance 5 mm.

EDS was used to study the surface layer composition of imaged samples using the FEI Helios NanoLab™ 660 (FEI). Each sample was analyzed at three locations and mapped for quantitative assessment. EDS was recorded at 10 keV with a dead time ranging from 40-60%. XPS analysis was conducted as described in the manufacturing and characterization section above.

Cell Culture on Functionalized Titanium Disks.

The cellular response to functionalized Ti disks was investigated by studying proliferation, and the expression of developmental genes and markers involved in mesodermal differentiation. Untreated Ti disks were used as controls. Following rehydration using ethanol solutions with decreasing concentrations, samples were transferred to 24-well plates and seeded with 1013A-MP cells (P6) at a density of 2 × 104 cells/ml in expansion medium as described above. 2 days after seeding, the samples were transferred to new 24-well ultra-low attachment plates, and cultured in osteogenic medium for additional 8 days.

Cell Growth.

To quantify cell growth, samples were incubated for 2 h with 10% (v/v) of PrestoBlue™ reagent (Life Technologies) in osteogenic medium. Following treatment, 200 μl of culture media were transferred to black, clear, flat-bottom 96-well plate (Corning). Fluorescence was measured at a 560 nm excitation and 590 nm emission using the fluorescent reader SYNERGYIVIx™ (BioTek®, Winooski, Vt.) equipped with Gen 5 1.09 software.

Gene Expression.

The expression level of developmental genes was investigated simultaneously using the NanoString nCounter™ system (Nanostring Technologies®, Seattle, Wash,), and data were analyzed with nSolver™ 2.5 software (see Table 51 for a list of all investigated genes). Briefly, samples were lysed in RLT buffer (Qiagen, Venlo, Nebr.) and total RNA extracted using the RNeasy™ Mini Kit (Qiagen) according to manufacturer's instructions. Extracted RNA was then quantified with the NanoDrop™ 8000 (Thermo Scientific) and 100 ng of samples were used for hybridization (65° C. for 18 h). The expression levels of investigated genes are expressed as fluorescent counts, in logarithmic scale, detected in the nCounter™ multiplex assay. Hierarchical cluster analysis was performed from normalized data using the RStudio™ package in R (available on the World Wide Web at R-project.org).

The expression of genes involved in mesodermal differentiation was studied via real-time PCR. Briefly, samples were washed in PBS and RNA was extracted as described above using the RNeasy™ Mini Kit (Qiagen). Purified RNA was quantified and reverse transcribed with random hex-amers using the GoScript™ Reverse Transcription System (Promega, Madison, Wis.) according to the manufacturer's protocol. Real-time PCR was performed using the StepOnePlus™ PCR System cycler (Applied Biosystems, Foster City, Calif.) in a 20 μl volume reaction using the TaqMan Universal PCR Master Mix and TaqMan Gene Expression Assays (Applied Biosystems) composed of FAM dye-labeled TaqMan MGB probe and PCR primers for runt-related transcription factor 2 (RUNX2; Hs00231692_ml), collagen, type I, alpha 1 (COL1A1; Hs00164004_ml), osteopontin (SPP1; Hs00959010_ml), SRY-Box 9 (SOX9; Hs00165814_ml), peroxisome proliferator activated receptor gamma (PPAR-γ; Hs01115513_ml), and VIC dye-labeled TaqMan MGB probe and primers for the housekeeping gene glyceraldehyde 3-phosphate dehydrogenase (GAPDH; Hs02758991_gl). The reactions consisted of a 95° C. cycle for 10 min followed by 40 cycles of denaturation (95° C. for 15 s) and annealing and extension (60° C. for 60 s). Results are expressed normalized to the expression levels of housekeeping gene GAPDH.

Alkaline Phosphatase Activity.

The activity of released alkaline phosphatase (ALP) was quantified using the Alkaline Phosphatase Activity Assay Kit™ (Biovision Inc., Milpitas, Calif.) according to the manufacturer's protocol. Reaction was conducted over a period of 150 min a RT in the dark. Fluorescence was measured at 450 nm using the plate reader SYNERGYMx™ (BioTek®, Winooski, Vt.) supplemented with Gen 5 1.09 software, and data normalized per content of cells as estimated using the Presto assay (described in section 2.7.1). ALP activity is expressed as “moles of PNP cleaved per reaction time”.

Statistical Analysis.

Prior to analysis, the data were tested for normality using the Prism software (Graphpad, La Jolla, Calif.). Gaussian distribution was detected using the Kolmogorov-Smirnov test. Significant differences were evaluated using ANOVA with Bonferroni correction for multiple comparisons. All results are shown as means and ±standard deviations. A difference between the mean values for each group was considered statistically significant when the P value was less than 0.05.

Results

Surface Topography and Composition of Titanium Disks.

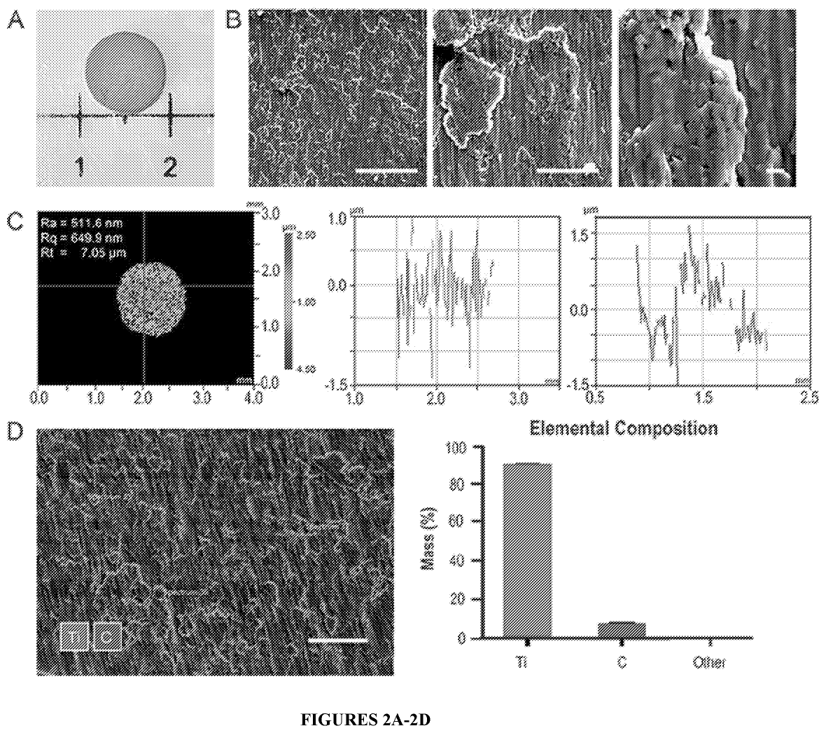

Topography and elemental composition of the Ti disks were studied using SEM, profilometry, EDS (FIG. 2), and XPS. SEM micrographs show that the Ti disks display a smooth topography with the presence of flake-like structures spread all across the disk area (FIG. 2B), and roughness parameters of Rₐ=511.6 nm, Rq=649.9 μm, and Rₜ=7.05 μm (FIG. 2C). Elemental analysis via EDS confirms the Ti composition of the disks and reveals the presence of traces of carbon in the samples (FIG. 2D). Other elements, including Si, Al, Mo, Ca, P, Na, and Fe, are present only in negligible amounts. XPS results acquired from the same samples show typical spectral peaks for Ti, C and O, as well as N, Ca and Si.

Cell Seeding, Distribution and Growth on Titanium Disks.

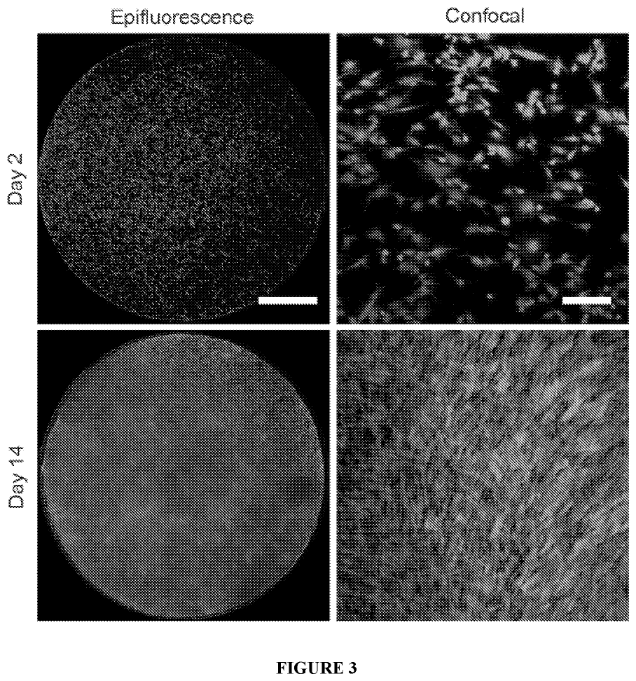

Cell seeding and growth on Ti disks were documented via live/dead assay. FIG. 3 shows that, 2 days after seeding, the cells are viable, display a healthy morphology, and are uniformly distributed over the disk area. After 14 days in culture under osteogenic conditions, cell proliferation leads to the formation of a monolayer tissue covering the entire disk area. Changes in cell morphology are also observed, indicating cell differentiation under induction culture conditions.

Characterization of Functionalized Titanium Disks.

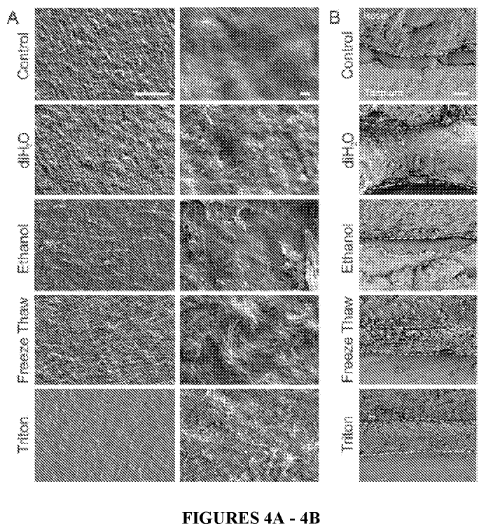

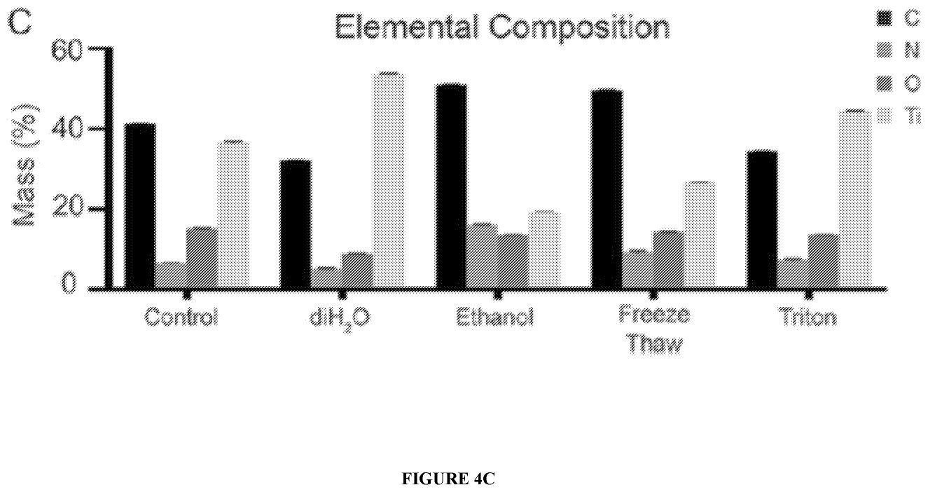

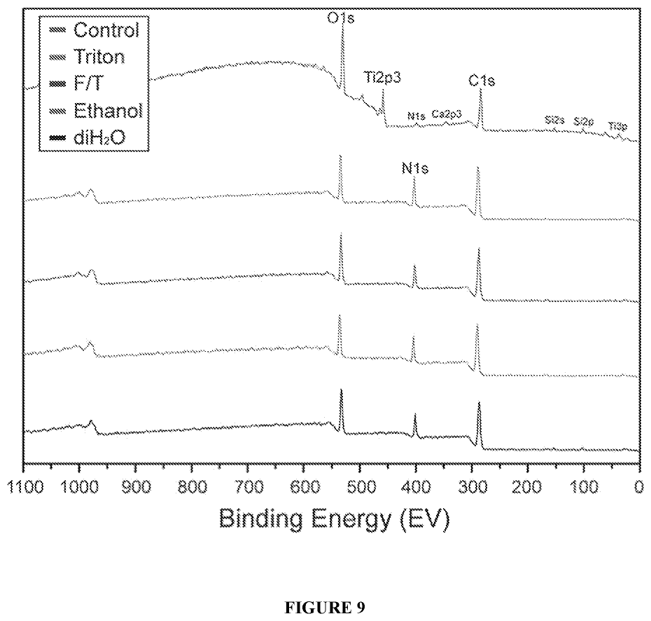

Following decellularization, the samples were characterized to study the ability of the different methods to lyse and remove the cells while preserving the integrity of the matrix. Non-decellularized samples were used as control. SEM imaging shows that different decellularization methods result in different surface modifications (FIG. 4). Treatment of the samples with diH₂O and repeated freeze thaw cycles does not seem to result in complete cell removal (FIG. 4A). It appears as though cell residuals may still be present on the surface of the disks. The topography of these samples, in fact, more closely resembles the topography of non-decellularized samples, where a dense layer of cells covers the surface of the Ti disks (FIG. 4A). On the other hand, treatment with ethanol and triton appear more efficient at washing out the cellular material and exposing a fibrous matrix (FIG. 4A). Interestingly, the appearance of this matrix is different, highlighting that different chemicals affect composition and integrity of the matrix differently. Whereas samples treated with ethanol display a matrix characterized by the presence of organized fibers of different size, samples treated with triton show a matrix with granular features and pores (FIG. 4A). Differences are also observed when the samples are imaged laterally (FIG. 4B). The results show that different decellularization methods result in the formation of biological coatings with different appearance and thickness. EDS analysis confirms functionalization of the Ti disks with biological material. FIG. 4B shows that both decellularized and non-decellularized samples are positive for C, N and O, indicating the presence of biological material covering the surface of the disks. Similar data are observed when the same samples are analyzed via XPS (FIG. 9). As opposite to control Ti disks, all decellularized samples display peaks for C, N and O, demonstrating the presence of biological material seating on the surface of the Ti disks.

Cell Response to Functionalized Ti Disks.

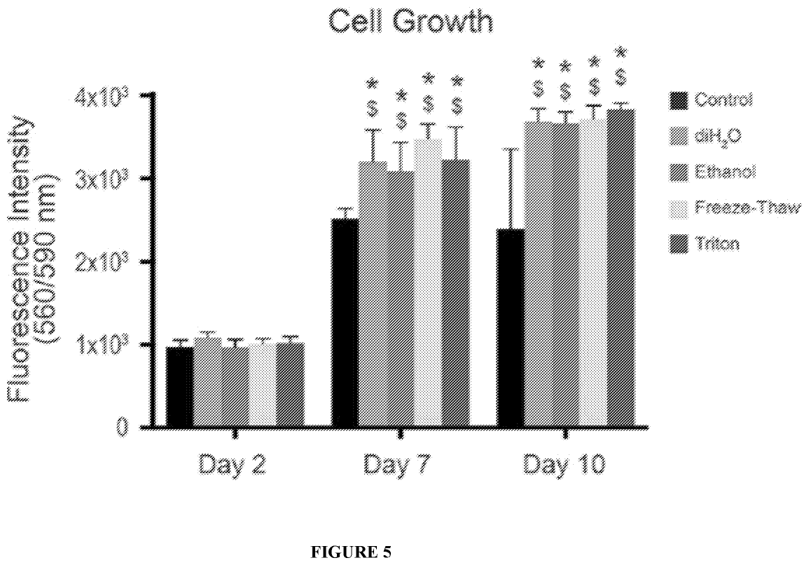

Following decellularization, the functionalized samples were seeded with fresh 1013A-MP cells to study the effect of the cell-mediated surface functionalization on cell proliferation, gene expression and release of alkaline phosphatase. Presto blue results (FIG. 5) show that, after 2 days, no significant differences in cell number exist between functionalized and control groups. However, after 7 and 10 days under induction culture condition, all functionalized disks display significantly increased number of cells compared to control group, suggesting that cell-mediated functionalization of Ti disks affect either survival or proliferation of 1013A-MP cells. Interestingly, despite different decellularization methods lead to different surface modifications, no significant differences are observed when comparing all four functionalized groups.

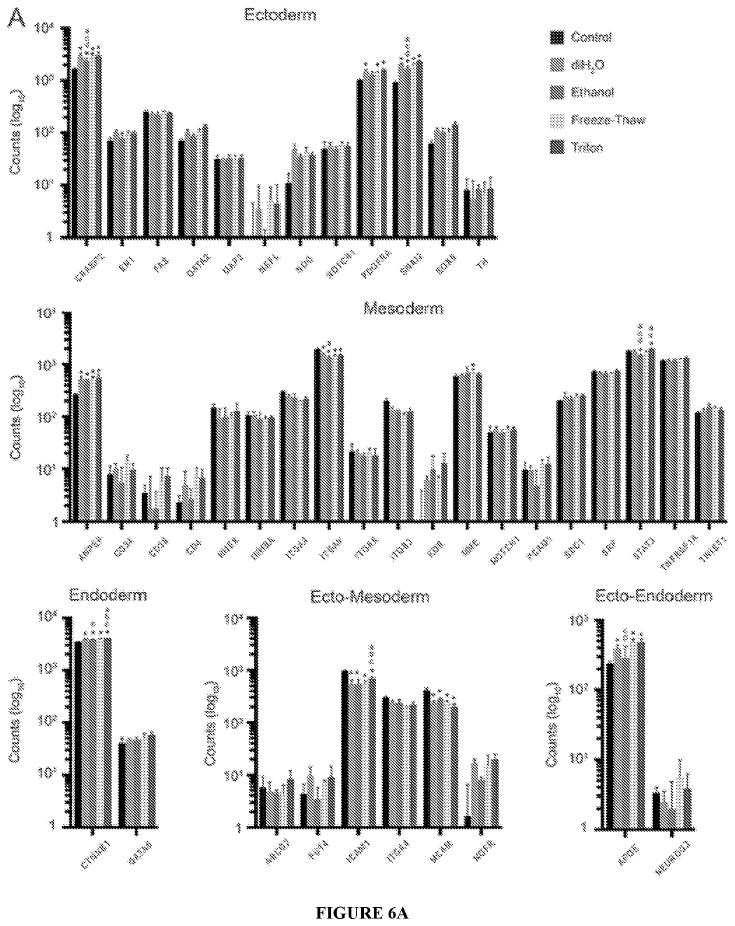

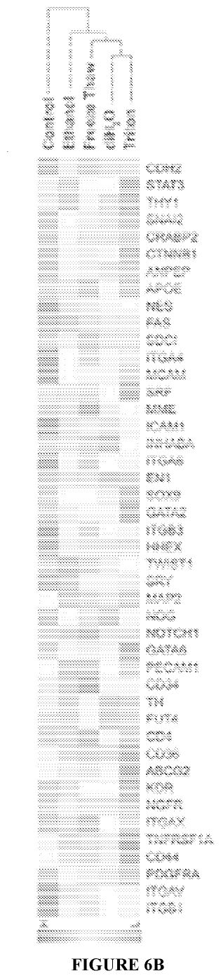

The expression of developmental genes was studied simultaneously using the Nanostring technology (FIG. 6). Overall, the results show that cell-mediated functionalization of Ti disks affect the expression of developmental genes in 1013A-MP cells. Out of 88 genes investigated, 44 genes are expressed when 1013A-MP cells are cultured on both functionalized and control groups. Among the expressed genes, 11 display significant differences when comparing functionalized and control groups. Of the genes involved in ectodermal differentiation, CRABP2, PDGFRA and SNAI2 show increased expression when cells are cultured on functionalized disks compared to control disks. Of the genes involved in mesodermal differentiation, ANPEP shows increased expression while ITGAV show decreased expression when cells are cultured on functionalized disks compared to control disks. Unlike these genes, WE shows increased expression only when cells are cultured on samples functionalized using the free-thaw method, whereas STAT3 shows increased expression only on samples functionalized using triton. For genes involved in endodermal differentiation, CTNNB1 displays increased expression when cells are cultured on the functionalized samples. For genes involved in the formation of more than one germ layer, the expression of ICAM1 decreases when cells are cultured on functionalized samples compared to the control group, whereas the expression of APOE increases. Interestingly, the expression of CRABP2, SNAI2, ITGAV, STAT3 and APOE is significantly lower when cells are cultured on samples decellularized using ethanol compared to samples decellularized with other methods. Hierarchical clustering results show that all functionalized groups cluster together, and are dissimilar from the control group (FIG. 6B).

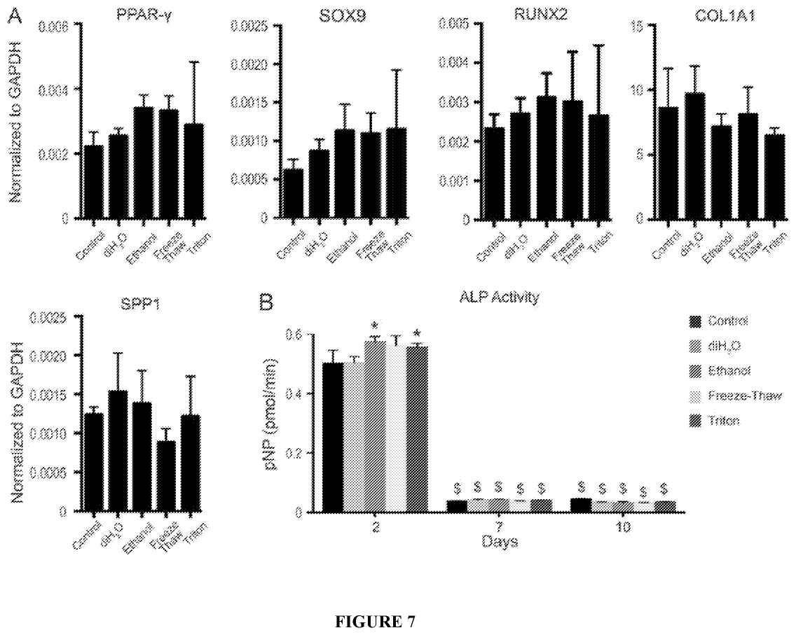

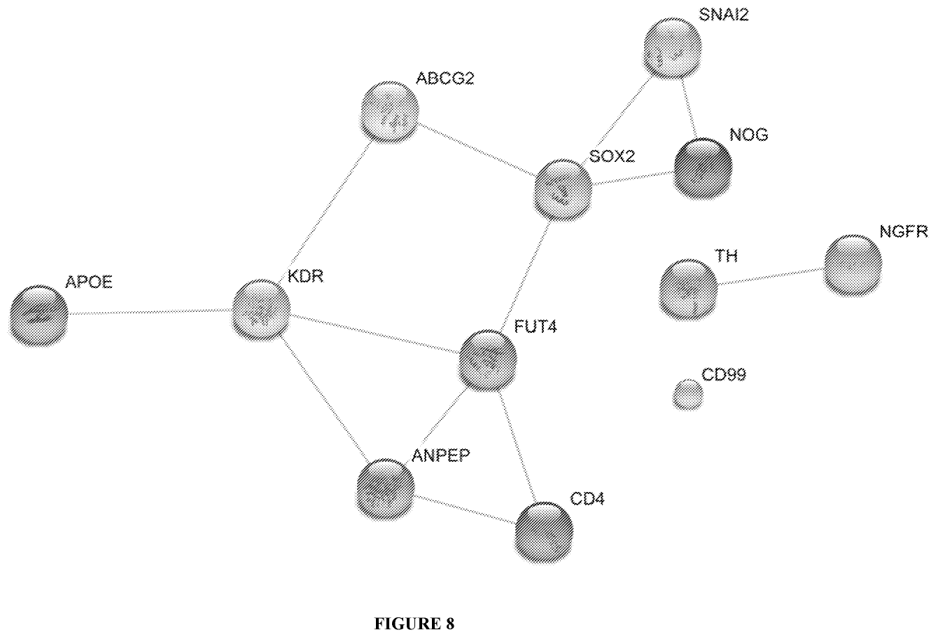

When studying the expression of mesodermal genes involved in adipogenic (PPAR-γ), chondrogenic (SOX9) and osteogenic (RUNX2, COLA1A and SPP1) differentiation, no significant differences are seen between functionalized and control groups (FIG. 7A). However, some trends are observed, with PPAR-γ, SOX9, RUNX2 and SPP1 showing increased expression and COL1A1 showing decreased expression when cells are cultured on the functionalized Ti disks.

The activity of ALP released in the medium was measured after 2, 7 and 10 days (FIG. 7B). The results show that, after 2 days, the cells cultured on samples functionalized using ethanol and triton release significantly higher amount of ALP than the cells cultured on the control group. After 7 days, ALP release drops for all groups and no significant difference are observed between functionalized and control groups. On the other hand, the activity of ALP measured per sample is higher after 2 and 7 days when cells are cultured on the functionalized disks compared to control (FIG. 10).

Selected Figure Legends

FIG. 2. Characterization of titanium disks. A—Photograph of a titanium (Ti) disk (diameter: 9 mm; area: 0.68 cm²). B—SEM images of a Ti disk taken at 1000× (scale bar=50 μm), 5000× (scale bar=10 μm), and 15,000× (scale bar=1 μm) magnification. C—3D morphological analysis of a Ti disk with values of surface roughness (Ra, Rq and Rt). D—EDS analysis of a Ti disk showing the surface elemental composition (scale bar=25 μm).

FIG. 3. Cell attachment, viability and growth on titanium disks. Epifluorescence micrographs (left, mosaic) and high magnification confocal images (right) showing distribution, viability and growth of human induced pluripotent stem cell-derived mesenchymal progenitors (1013A-MP) on titanium disks 2 and 14 days after seeding. Samples were stained with the Live/Dead assay (green=live cells; red=dead cells). Scale bars=2 mm and 100 μm respectively.

FIG. 4. Characterization of functionalized titanium disks. A—SEM images of non-decellularized (control) and decellularized samples taken at 1000× (scale bar=50 μm), and 15000× (scale bar=1μm) magnification. B—Side-view SEM images of non-decellularized (control) and decellularized samples showing the engineered biological coatings in between the titanium disk (Ti) and the polymethyl methacrylate (resin). Scale bar=10 μm. C—EDS analysis of non-decellularized (control) and decellularized samples confirming the presence of biological material on the surface of the titanium disks.

FIG. 5. Cell growth. Quantification using the PrestoBlue™ assay of human induced pluripotent stem cell-derived mesenchymal progenitors (1013A-MP) cultured onto functionalized and control titanium disks. Data represent averages±SD (2-way ANOVA with Bonferroni's multiple comparisons test, n=4, alpha=0.05, P<0.0001; * denotes significant difference to control samples; $, significant difference to day 2).

FIG. 6. Expression of developmental genes. A—Nanostring analysis of human induced pluripotent stem cell-derived mesenchymal progenitors (1013A-MP) 10 days after culture on functionalized and control titanium disks. Only expressed genes are shown (44 out of 88). Data represent averages±SD (2-way ANOVA with Bonferroni's multiple comparisons test, n=3, alpha=0.05, P<0.0001; * denotes significant difference to control samples; a, significant difference to diH2O; b, significant difference to ethanol; c, significant difference to freeze-thaw; d, significant difference to triton). B—Hierarchical clustering of cells seeded on decellularized samples and control groups. Pink represents high expression and blue represents low expression when compared to normalized threshold values.

FIG. 7. Expression and production of mesodermal markers. A—Expression of genes involved in osteogenic, chondrogenic and adipogenic differentiation for human induced pluripotent stem cell-derived mesenchymal progenitors (1013A-MP) cultured on functionalized and control titanium disks. Data represent averages±SD (ANOVA with Bonferroni's multiple comparisons test, n=4, alpha=0.05, P=0.3651, 0.3282, 0.8297, 0.2361 and 0.2227 respectively). B—Alkaline phosphatase (ALP) release from 1013A-MP cells cultured on functionalized and control titanium disks. Data represent averages±SD (2-way ANOVA with Bonferroni's multiple comparisons test, n=3, alpha=0.05, P=0.0005; * denotes significant difference to control samples; $, significant differences to day 2).

Discussion

Ti prosthetic implants are widely used in reparative dentistry and orthopedics. Following implantation into a bone cavity, and under desirable healing conditions, these devices become fully integrated with the surrounding tissue reestablishing function. However, osseointegration is a highly organized biodynamic process that takes time, fails in at-risk patients with compromised regenerative ability, and can result in complications such as loosening and infection. Thus, intense research efforts are ongoing both in academia and industry to functionalize the surface of prosthetic implants with features that favor the integration process. Physical and chemical modifications of prosthetic implants change the surface energy and osteoconductivity properties of these devices—by modulating for example the adhesion of body fluid molecules and increasing the bone-implant contact area—but cannot actively control cell response and tissue regeneration. In order to better modulate the surrounding tissue environment, researchers have lately attempted to functionalize the surface of prosthetic implants via immobilization of ‘biologically active’ molecules (e.g. ECM proteins, cytokines, growth factors) or incorporation of stem cells with encouraging results. Nevertheless, immobilization of biologically active molecules (in their functional form) is difficult, may require surface activation, and cannot resemble the complex molecular organization typical of native tissues. On the other hand, incorporation of stem cells has proven to induce healing and promote bone formation but carry potential clinical risks associated with unforeseen cell behavior. This is especially true if the cells are manipulated ex vivo or are derived from pluripotent stem cells via cloning or reprogramming. New strategies must therefore be developed to functionalize prosthetic implants with coatings of high biological relevance that are safer, can be individualized and display enhanced osteoconductive and osteoinductive properties.

Decellularization methods allow researchers to remove cells from tissue and organs while preserving the mixture of structural and functionally active proteins that constitute the ECM. In light of this knowledge, the inventors have explored the possibility of using stem cells to engineer biological coatings displaying the molecular complexity typical of native tissues. Model Ti disks with standard surface topography and chemistry were seeded with 1013A-MP cells (mesenchymal progenitors generated from dermal fibroblasts using Sendai virus), and samples cultured under osteogenic induction conditions until the formation of a uniform monolayer tissue covering the surface of the disks. Human iPS cells can be derived from every patient in large amounts, and can give rise to all cells constituting the human body, thus enabling the engineering of biological coatings for diverse biomedical applications.

Several decellularization methods exist, including physical, chemical and enzymatic methods, and combination thereof. Each method is recognized to affect the biochemical composition and integrity of the remaining extracellular matrix, which in turn may influence the response of cells and tissues exposed to it. To test this hypothesis, in this study the decellularization ability of physical (distilled water and freeze-thaw cycles) and chemical (ethanol and triton) methods was compared, as well as the effect of method-specific functionalization on cell behavior in vitro. Each method was chosen and properly adapted with the objective of minimizing cost and limiting the use of toxic chemicals that could hamper translation and commercialization.

Snap freezing in a wet environment can disrupt the cell membrane through the formation of intracellular and extracellular ice crystals. However, this process can also affect ECM integrity and properties.

Treatment with distilled water results in an osmotic shock and cell swelling, leading to disruption of the cell membrane. While it can preserves the integrity of the ECM, this method cannot remove the cellular material easily, resulting in the formation of biological coatings that potentially retain immunological properties.

Ethanol is a volatile solvent that can be used to delipidize and sterilize the samples at the same time. On the other hand, sample dehydration associated with treatment can affect the integrity and mechanical properties of the ECM, and reduce its functional properties.

Triton is a non-ionic detergent that disrupts the lipid-lipid and lipid-protein interactions but leaves the protein-protein interactions intact. It is known not to substantially affect the ECM integrity but can reduce the glycosaminglycan content.

In this study, SEM, EDS and XPS characterization of functionalized Ti disks demonstrated that the different treatments have different decellularization potential and result in the formation of biological coatings displaying different characteristics. Osmotic shock with diH₂O did not seem to efficiently remove the cells; it appears as though cellular remnants may still be present on the surface of the Ti disks. SEM analysis demonstrates that the topography of these diH₂O-treated samples more closely resembles the topography of non-decellularized samples, where a smooth landscape of cells covers the surface of the Ti disks. Not even freeze-thaw treatment in wet environment appeared to efficiently remove the cell content, but lateral imagining reveals the presence of pores possibly resulting from ice crystals formed during the freeze-thaw process. Treatment with ethanol and Triton seem to better wash out the cellular material and expose the matrix. Interestingly, the appearance of this matrix is different between these two treatments, which is in line with the fact that different chemicals affect composition and integrity of the matrix in different ways. Whereas samples treated with ethanol display a matrix characterized by the presence of organized fibers of different size, samples treated with triton show a matrix with granular features and pores. However, it is not yet clear how the different treatments affect the content of lipids and proteins. Additional characterization studies are needed to elucidate the composition of stem cell-engineered coatings and drive protocol optimization. To avoid substantial tissue disintegration or detachment from the surface of the Ti disks, in this study each decellularization treatment was applied for a reduced period of time and under mild agitation. It is likely that variation in the number and duration of each decellularization cycle could lead to better outcomes, and optimization studies are critical for further development and translation of implants functionalized using this strategy. To study the biological effect of cell-mediated functionalization, following decellularization the samples were seeded with fresh 1013A-MP cells and the constructs were cultured for 10 days.

The results show that, irrespective of the decellularization method used, all functionalized groups influenced proliferation, gene expression and release of the bone marker ALP. Interestingly, cell growth significantly increased when cells were cultured on the functionalized disks compared to control disks. Functionalization also influenced the expression of specific genes involved in germ layer specification, with some genes significantly up-regulated (CRABP2, PDGFRA, SNAI2, ANPEP, CTNNB1 and APOE) and some significantly down-regulated (ITGAV, ICAM1, and MCAM) when cells were cultured on the on the functionalized disks compared to control disks. As opposed to cell growth, the level of expression of some of these genes displayed significant difference also among samples decellularized using different methods, indicating that each method produces biological coatings with different functional properties. The effect of surface functionalization on gene expression was also observed for other genes, including mesodermal genes. Although these differences were not significant, PPAR-γ, SOX9, RUNX2 and SPP1 displayed increased expression and COL1A1 decreased expression when 1013A-MP cells were cultured on functionalized disks compared to control disks. Increase in the expression of these genes suggests that the biological coatings engineered using this strategy could promote osseointegration following implantation in vivo. In line with this, 2 days after seeding, the cells released more ALP when cultured on samples functionalized following treatment with ethanol and triton compared to control group. ALP is an early marker of osteogenic differentiation, which is responsible for the mineralization of the ECM. It is interesting to see that this happens during the first days of culture, when the cells are kept under expansion culture conditions (i.e. in absence of osteoinductive factors). An increase in ALP release and activity at the implantation site could facilitate ossification of the tissue-implant interface and consolidate implant integration and stability.

In summary, the inventors have successfully used stem cells to functionalize the surface of prosthetic implants with biological coatings of high molecular complexity. The results show that stem-cell based functionalization of Ti implants affect cell growth, gene expression and release of ALP, and could lead to the development of a new generation of prosthetic implants with enhanced therapeutic properties. Interestingly, the effect of stem cell-mediated functionalization appears to be treatment specific, thus suggesting that protocol optimization could lead to development of biological coatings with increased functional properties. Important questions therefore remain and further studies are needed to realize the potential of this technology. Developing optimal cell culture and decellularization protocols, assessing the response of different cell types to functionalized implants (i.e. the cells playing a role during the different phases of the osseointegration process), understanding the relationship between implant surface characteristics and quality of the coating, and in vivo testing are all important aspects that must be considered before cell-mediated functionalization can be employed to enhance the therapeutic potential of prosthetic implants.

Conclusion

Biofunctionalization of prosthetic implants is expected to forward development of devices with enhanced therapeutic potential. In this study the inventors have demonstrated that human stem cells can be used to engineer coatings of high biological complexity and distinct functional properties. The technology opens the possibility to develop a new generation of prosthetic implants that are safer, meet the regulatory requirements, and can be individualized for advanced dental and orthopedic reconstructions.

Although the invention has been described with reference to the above examples, it will be understood that modifications and variations are encompassed within the spirit and scope of the invention. Accordingly, the invention is limited only by the following claims.

1. A method for functionalizing a surface of an implant to promote osseointegration upon implantation into bone tissue, comprising:
a) seeding the surface of the implant with mesenchymal progenitor (MP) cells;
b) culturing and expanding the cells to produce an extracellular matrix (ECM) on the surface; and
c) decellularizing the surface, thereby functionalizing the surface of the implant.

2. The method of claim 1, wherein decellularizing removes cells from the surface while maintaining the ECM.

3. The method of claim 2, wherein decellularizing comprises incubating the surface in a treatment solution comprising water, an alcohol, or a nonionic surfactant.

4. The method of claim 3, wherein the treatment solution consists of deionized water.

5. The method of claim 3, wherein the treatment solution is a mixture of ethanol and water.

6. The method of claim 3, wherein the treatment solution comprises a nonionic surfactant having the formula:

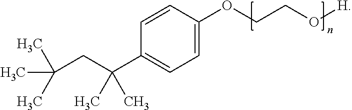

7. The method of claim 6, wherein the nonionic surfactant is polyethylene glycol tert-octylphenyl ether (Triton X-100™).

8. The method of claim 3, wherein the surface is incubated at 37° C. for about 30 to 60 minutes.

9. The method of claim 1, wherein decellularizing comprises freezing the surface in a physiological buffer.

10. The method of claim 9, wherein the physiological buffer is phosphate-buffered saline (PBS).

11. The method of claim 9, wherein the surface is frozen for greater than 30 minutes at −80° C. and subsequently thawed.

12. The method of claim 1, further comprising treating the surface with a nuclease after decellularizing.

13. The method of claim 12, wherein the surface is treated with a DNAse and an RNAse.

14. The method of claim 12, further comprising dehydrating the surface with successive applications of an ethanol-water solution, in which each successively applied solution has a higher concentration of ethanol, the final application being application of 100% ethanol.

15. The method of claim 1, further comprising coating the surface with at least one molecule that supports adherence of a living cell before seeding the surface with MP cells.

16. The method of claim 15, wherein the molecule is selected from the group consisting of polypeptide, gelatin, matrigel, entactin, glycoprotein, collagen, fibronectin, laminin, poly-D-lysine, poly-L-ornithine, proteoglycan, vitronectin, polysaccharide, hydrogel, and combinations thereof

17. The method of claim 1, further comprising reseeding the surface after decellularization, reseeding comprising seeding the surface with MP cells.

18. The method of claim 17, further comprising culturing and expanding the reseeded MP cells.

19-24. (canceled)

25. An implant produced by the method of claim 1.

26-45. (canceled)

46. A method for performing a medical procedure comprising:
a) obtaining a cell from a subject;
b) reprogramming the cell of (a) to generate an inducted pluripotent stem (iPS) cell;
c) differentiating and expanding the iPS cell to generate mesenchymal progenitor (MP) cells;
d) seeding the MP cells onto the surface of an implant;
e) culturing and expanding the MP cells to produce an extracellular matrix (ECM) on the surface;
f) decellularizing the surface, thereby functionalizing the surface of the implant to promote osseointegration upon implantation into bone tissue; and
g) implanting the implant into bone tissue of the subject.

47-69. (canceled)
